(19) United States
(12) Patent Application Publication
(10) Pub. No.: US 2025/0325753 A1
(43) Pub. Date: Oct. 23, 2025

(54) SYRINGE AND SYRINGE SYSTEM FOR MANIPULATING A FLUID

(71) Applicant: Life Technologies Corporation, Carlsbad, CA (US)

(72) Inventor: Michael Gordon, Encinitas, CA (US)

(21) Appl. No.: 18/870,090

(22) PCT Filed: Jun. 1, 2023
(86) PCT No.: PCT/US2023/067750

§ 371 Date: Nov. 27, 2024

Related U.S. Application Data
(60) Provisional application No. 63/348,932, filed on Jun. 3, 2022.
(60) Provisional application No. 63/448,998, filed on Feb. 28, 2023.

(51) Int. Cl. A61M 5/31; A61M 5/315

(52) U.S. Cl. CPC A61M 5/3135; A61M 5/31505; A61M 5/31515; A61M 2005/3114

(57) ABSTRACT

Apparatus, methods, and systems including a syringe for delivering a fluid. The syringe includes a syringe body, a piston, an insert, and a removable plunger. The piston is located in a lumen of the syringe body such that a bottom of the piston together with interior walls of the syringe define a working volume. The piston comprising a cavity. The insert is located at a desired position in the lumen. The insert narrowing the lumen and being configured to prevent retraction of the piston beyond the desired position. The removable plunger is configured to removably couple to the piston. The removable plunger includes a tip configured to couple with the cavity of the piston and move the piston when a force is applied at the plunger, and to decouple from the piston when the piston engages the insert at the desired position.

### FIELD

This disclosure relates generally to fluid delivery, and more particularly to improvements to syringes, as well as methods of using syringes, aspirating syringes, and systems employing the syringes for manipulation of biological samples.

### BACKGROUND

Syringes are popularly used for the manipulation of samples in research, medical, and diagnostic settings. A syringe typically consists of a needle attached to one end of a hollow cylinder and a plunger connected to a sliding piston. Fluid is drawn into the hollow cylinder from the needle by pulling on the plunger. Fluid can be injected or dispensed from the hollow cylinder by pushing on the plunger causing the piston to push out the fluid from the needle. The syringes can be filled with desired sample just before using the syringe, or the syringes can be pre-filled with desired sample.

In several applications, pre-filled syringes transport samples from one point to another (e.g., within a laboratory or one geographical location to another). Such pre-filled syringes have plungers attached to the piston that typically extend well beyond the top of the hollow cylinder. When the plunger is accidentally pushed or bumped, the fluid can dispense at an undesired time and/or location. Thus, there exists a need for improved syringes that enable transport of syringes without the risk of accidental discharge.

### BRIEF SUMMARY

One aspect of the present disclosure relates to a syringe. A syringe includes a syringe body having an inlet and interior walls defining a lumen having a longitudinal axis and a diameter, a piston located in the lumen of the syringe body, an insert located at a desired position in the lumen, and a removable pull rod, also referred to, and used interchangeably herein with removable plunger. The piston has a top and a bottom, the bottom of the piston together with the interior walls defining a working volume. The piston includes a cavity. The piston is movable along the longitudinal axis to change the working volume. The insert narrows the lumen and prevents retraction of the piston beyond the desired position. The removable pull rod is configured to removably couple to the piston. The removable pull rod includes a tip that couples with the cavity of the piston and moves the piston inside syringe body when a force is applied at the removable pull rod. The removable pull rod decouples from the piston when the piston engages the insert at the desired position.

In some embodiments, the tip is conical in shape having a narrow end configured to enter the cavity in the piston and a base end configured to couple the piston to the tip. The base end of the tip has a chamfered, spherical, or fillet edge configured to facilitate retraction of the tip from the cavity of the piston. The base end includes a base portion configured to taper away from the narrow end. The piston is made of a compliant material.

In some embodiments, the cavity is cylindrical in shape and sized to provide a friction fit between the tip of the removable pull rod and the piston. The cavity is sized to receive the tip and tightly fit around the tip such that the piston is retractable by the removable pull rod. The cavity of the piston is configured to cover the base portion of the tip.

In some embodiments, the piston is made of a compliant material. The cavity of the piston is sized smaller than a size of the tip so as to create a friction fit with the tip of the removable pull rod. When the piston receives the removable pull rod, the cavity of the piston expands around the tip thereby coupling the removable pull rod and the piston. The piston is disposed at a proximal end of the syringe configured to receive the fluid, and the desired position is at a distal end of the syringe indicative of a desired amount of the fluid to be filled in the syringe. The cavity extends partially toward the bottom of the piston but does not extend through the bottom of the piston. The piston is disposed axially in the syringe body, and the cavity is formed at a center of the piston and extends axially.

In some embodiments, the insert includes a hollow cylinder, optionally including a flange, barb, recess or groove, defining an internal channel, or can be ring shaped formed from a plastic of the syringe. The internal channel is larger than an outside diameter of the removable pull rod and is configured to allow the removable pull rod to movably extend through the internal channel and move along the longitudinal axis, and to remove the removable pull rod from the syringe when decoupled from the piston. The insert is removably inserted at a distal end of the syringe opposite the inlet. In another example, the insert is fixedly attached at a distal end of the syringe at the desired position in the lumen.

Further, one aspect of the present disclosure relates to a method for aspiration of a syringe, the syringe comprising a piston and a removable pull rod. The method includes inserting the removable pull rod from a distal end of the syringe into a lumen defined by interior walls of the syringe to couple with the piston, retracting the removable pull rod to retract the piston from a proximal end of the lumen toward the distal end causing a fluid to enter the syringe via an inlet in the proximal end, preventing further retraction of the piston via an insert positioned within the lumen; and removing the removable pull rod from the piston.

In some embodiments, the inserting the removable pull rod involves aligning a tip of the removable pull rod with a cavity of the piston; and pushing the tip into the cavity until the removable pull rod is coupled with the piston. The tip is configured to couple with the cavity of the piston to move inside syringe body when a force is applied at the removable pull rod and is configured to decouple from the piston when the piston engages with the insert.

In some embodiments, removing the removable pull rod involves further retracting the removable pull rod while the piston is engaged with the insert, thereby removing the tip of the removable pull rod from the cavity of the piston such that the piston stays inserted in the syringe, while the removable pull rod is completely detached from the piston.

Further, the method involves securing the insert at a desired position in the lumen, the desired position corresponding to an amount of fluid to be filled in the syringe.

Further, one aspect of the present disclosure relates to a system employing one or more syringes for dosing and delivering a desired amount of fluid. The system includes a syringe, a feedline to deliver contents of the syringe, and a syringe receptacle. The syringe includes a syringe body having interior walls defining a lumen having a longitudinal axis, a removable pull rod comprising a tip, a piston located in the lumen of the syringe body, the piston together with the interior walls defining a working volume configured to hold fluid, the piston comprising a cavity configured to engage with the tip of the removable pull rod, and an insert narrowing the lumen to prevent retraction of the piston beyond a desired position. The feedline delivers droplets from the syringe into a sterile, fluidic system. The syringe receptacle receives the syringe and fluidically couples the syringe to the feedline. The system forms a sterile and functionally-closed system which can be utilized, e.g., in a workflow to produce a cell therapy product.

In some embodiments, the receptacle and the syringe are coupled together by a fastening mechanism, such as a snap, latch or threaded engagement, disposed at a distal end of the syringe. For example, in some embodiments, the receptacle and the syringe are coupled by a threaded engagement, wherein the syringe includes threads formed at a proximal end on an outer surface of the syringe, and the receptacle includes corresponding threads at an interior portion of the receptacle. The receptacle includes a pierceable septum to be penetrated by a needle attached at a proximal end of the syringe such that a tip of the needle enters the feedline to supply droplets into the feedline. The receptacle includes a plurality of hollow chambers, each chamber configured to receive one syringe. One or more hollow chambers of the receptacle are configured to receive one or more syringes of different sizes. The feedline has an inlet and an outlet, the receptacle being connected between the inlet and the outlet, wherein the inlet receives a fluid, such as sterilized gas, air, or liquid (e.g., liquid buffer), from a pump and the outlet delivers fluids from the plurality of syringes into the receptacle.

In some embodiments, the system further includes a delivery mechanism configured to inject, at a specified time and a specified amount, fluid from one or more syringes in the receptacle.

Further, one aspect of the present disclosure relates to a method of dispensing contents of a syringe. The method involves filling a syringe by retracting a removable plunger coupled to a piston located within a lumen of a syringe body, separating the removable plunger from the piston after filling the syringe, coupling of a syringe receptacle and a feedline, inserting the plungerless syringe into the syringe receptacle to fluidically couple the syringe to a feedline, coupling the plunger to the piston, and advancing the plunger by a specified amount to move the piston within the lumen of the syringe body and thereby deliver a desired amount of fluid into the feedline. The fluidically coupling of the syringe to the feedline forms a sterile and functionally-closed system.

The forgoing general description of the illustrative implementations and the following detailed description thereof are merely exemplary aspects of the teachings of this disclosure, and are not restrictive.

### BRIEF DESCRIPTION OF THE DRAWINGS

The accompanying drawings, which are incorporated in and constitute a part of the specification, illustrate one or more embodiments and, together with the description, explain these embodiments. The accompanying drawings have not necessarily been drawn to scale. Any values dimensions illustrated in the accompanying graphs and figures are for illustration purposes only and can or cannot represent actual or preferred values or dimensions. Where applicable, some or all features cannot be illustrated to assist in the description of underlying features. In the drawings:

FIG. 1A illustrates one embodiment of an empty syringe;

FIG. 1B illustrates one embodiment of a syringe containing liquid;

FIG. 1C illustrates one embodiment of a tip of a plunger or pull rod that engages with a piston of the standard piston of FIGS. 1A and 1B;

FIG. 2A depicts one embodiment of an exemplary syringe with a detached plunger;

FIG. 2B is a cross section view of one embodiment of a plunger tip engaged with the piston of the syringe;

FIG. 3 illustrates a geometry of one embodiment of the plunger tip that engages with the piston of the syringe;

FIG. 4A is a perspective view of one embodiment of an insert used with the syringe;

FIG. 4B is a cross section view of one embodiment of the insert of FIG. 4A;

FIG. 4C is a cross section view of one embodiment of the insert of FIG. 4A engaging with the piston of the syringe;

FIGS. 5A-5D illustrates a method of loading sample into a syringe as provided herein, and removal of the plunger from the syringe;

FIG. 6A is an elevated perspective view illustrating one embodiment of an exemplary multi-channel syringe system employing the syringes of FIGS. 2-4, the multi-channel syringe system being configured to inject fluid from one or more syringes at a desired time and at a desired amount;

FIG. 6B is front view of one embodiment of the multi-channel syringe system of FIG. 6A that can be connected to a pump or vacuum and is configured to receive a sterilized fluid, such as liquid, gas or air, mix the fluids in the multi-channel syringe system, and deliver the mixed fluids to a system, e.g., functionally-closed system;

FIG. 6C is a cross section view of one embodiment of the multi-channel syringe system of FIG. 6A;

FIG. 6D is an isometric view of a syringe receptacle of the multi-channel syringe system;

FIG. 6E is an isometric view of a feedline that can be coupled to the syringe receptacle;

FIG. 7A is an elevated perspective view illustrating one embodiment of an exemplary multi-channel syringe system employing the syringes of FIGS. 2-4, the multi-channel syringe system being configured to inject fluid from one or more syringes at a desired time and at a desired amount;

FIG. 7B is a front view of one embodiment of the multi-channel syringe system of FIG. 7A that can be connected to a pump or vacuum and is configured to receive a sterilized fluid, such as liquid, gas or air, mix the fluids in the multi-channel syringe system, and deliver the mixed fluids to a system, e.g., functionally-closed system;

FIG. 7C is a front view showing internal components of the multi-channel syringe system of FIG. 7A;

FIG. 7D is a cross section front view of the multi-channel syringe system depicted in FIG. 7A;

FIG. 7E is an isometric cross section view of the multi-channel syringe system depicted in FIG. 7A;

FIG. 7F is a perspective view of the multi-channel syringe system depicted in FIG. 7A prior to insertion of the syringes into the receptacle;

FIG. 7G is a front view of the multi-channel syringe system depicted in FIG. 7A prior to insertion of the syringes into the receptacle showing internal components; and

FIG. 7H is a cross section front view of the multi-channel syringe system depicted in FIG. 7A prior to insertion of the syringes into the receptacle.

### DETAILED DESCRIPTION

The description set forth below in connection with the appended drawings is intended as a description of various embodiments of the disclosed subject matter and is not necessarily intended to represent the only embodiment(s). In certain instances, the description includes specific details for the purpose of providing an understanding of the disclosed embodiment(s). However, it will be apparent to those skilled in the art that the disclosed embodiment(s) can be practiced without those specific details. In some instances, well-known structures and components can be shown in block diagram form in order to avoid obscuring the concepts of the disclosed subject matter.

Reference throughout the specification to “one embodiment” or “an embodiment” means that a particular feature, structure, or characteristic described in connection with an embodiment is included in at least one embodiment of the subject matter disclosed. Thus, the appearance of the phrases “in one embodiment” or “in an embodiment” in various places throughout the specification is not necessarily referring to the same embodiment. Further, the particular features, structures or characteristics can be combined in any suitable manner in one or more embodiments. Further, it is intended that embodiments of the disclosed subject matter cover modifications and variations thereof.

It is to be understood that terms such as “distal,” “proximal,” “top,” “bottom,” “front,” “side,” “length,” “lower,” “interior,” “inner,” “outer,” and the like that can be used herein merely describe points of reference and do not necessarily limit embodiments of the present disclosure to any particular orientation or configuration. Furthermore, terms such as “first,” “second,” “third,” and the like, merely identify one of a number of portions, components, steps, operations, functions, and/or points of reference as disclosed herein, and likewise do not necessarily limit embodiments of the present disclosure to any particular configuration or orientation.

The terms “longitudinal,” “axial” or “axially” are generally longitudinal as used herein to describe the relative position related to a syringe, a delivery mechanism, and components of the system herein. The term “radial” generally refers to a direction perpendicular to the “axial” direction. For example, the term “radial” generally refers to a direction perpendicular to the axis “A”.

The term “fluid” refers primarily to a liquid, but also includes a suspension of solids diffused in the liquid, dissolved therein, or gas that coexists in the liquid inside the fluid-containing portion of the syringe. In the present disclosure, fluid is used as an example substance aspirated into a syringe for explaining the concepts. In many embodiments, samples can be aspirated without deviating from the scope of the present disclosure.

As used herein, the term “sample” refers to a liquid which can be used in an assay, such as a chemical or biological assay, and may include one or more reagents, such as a biological molecule.

The term “biological molecule” or “biomolecule” is intended to generally refer to any organic or biochemical molecule that occurs in a biological system including a whole cell, a cellular component, a substrate, or any portions thereof.

As used herein, a “cellular component” is intended to include any component of a cell that may be at least partially isolated upon lysis of the cell. Cellular components include components that are recombinantly or synthetically produced which may be functionally and/or structurally altered to include synthetically (e.g., chemically synthesized) derived components. Cellular components may be organelles, such as nuclei, perinuclear compartments, nuclear membranes, mitochondria, chloroplasts, or cell membranes; polymers or molecular complexes, such as lipids, polysaccharides, proteins (membrane, trans-membrane, or cytosolic); nucleic acids, viral particles, or ribosomes; or other molecules, such as hormones, ions, cofactors, or drugs.

In various embodiments, a sample includes a cell (e.g., a mammalian cell or a non-mammalian cell), cellular component, biomolecule, or other reagent (e.g., a reagent used in the development or manufacturing of cell and gene therapies). In some embodiments, the sample can be a fluid and loaded into a syringe through a tube, a vial, or other container(s). In some embodiments, the tube, vial or other container is single-use and composed of a material suitable for preparing, mixing, centrifuging, transporting and/or storing solid and liquid samples and reagents, such as quartz, glass, metal or a polymer (e.g., polypropylene, polyvinyl, polyurethane, polycarbonate and the like).

Systems used in cell-based or gene-based therapies (e.g., CAR T-cell therapy) involve numerous pieces of equipment performing multiple processes, and the addition of numerous reagents/cell culture components to various pieces of equipment, often in a specified sequence.

In the field of cell and gene therapy, functionally-closed, sterile systems are highly desirable. “Open” steps (e.g., involving manual intervention) should be minimized to reduce the risk of biological contamination.

Presently, when electroporation, a common technique used in gene editing, is used to deliver a payload (e.g., biomolecule, such as a nucleic acid or protein, or other reagent), cells are concentrated and resuspended in electroporation buffer (e.g., electrically conductive fluid) within a bag or rigid vessel. Once resuspended, additional reagents can be added to the cells in a specific sequence. At present time, this is achieved within a biosafety cabinet and is considered to be an open step, which carries some risk of contamination. Ideally, this step occurs outside of a biosafety cabinet and within an automated closed system, however, current solutions for automatic injection of different fluids are inadequate, especially for low volume fluids (e.g., <1 ml). Following the addition of the payload and requisite additional reagents, the bag or other storage medium containing the cells is then coupled to an electroporation platform to electroporate the cells.

Arrangement of syringes and automatic injection of fluid can be associated with problems including, for example, dead volumes, priming issues, or the accidental injection of liquid such as, for example, when a plunger is bumped or inadvertently depressed.

The present disclosure solves several problems outlined above, and provides new systems, methods, and devices for injection of samples (e.g., including fluids, suspensions, and the like). In some embodiments, for automating the injection of different samples, a plurality of syringes can be disposed in a casing (e.g., consumable) to inject different samples in a tubing connecting two instruments. Plungers of one or more syringes can be manipulated to dispense a desired amount of sample from the syringes at the desired time, and into the desired location.

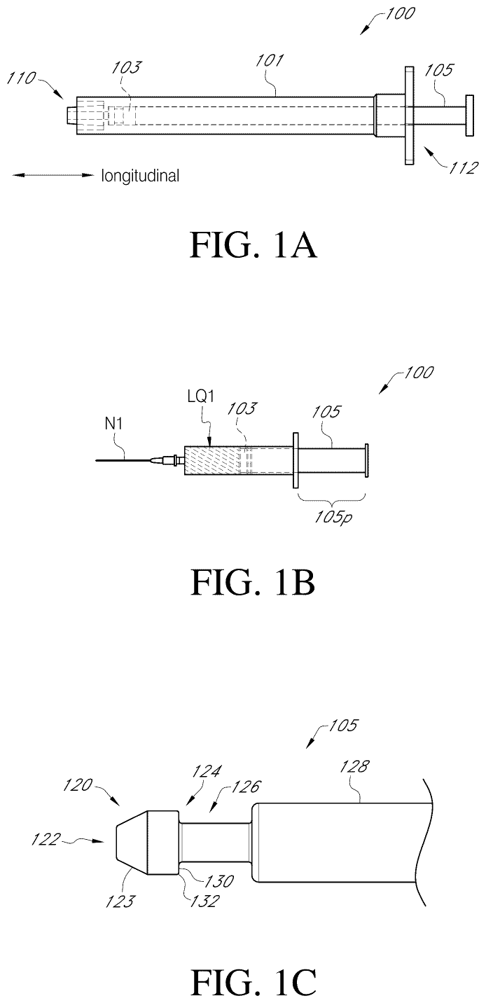

FIGS. 1A-1C illustrate one embodiment of a syringe 100. Syringe 100 can be a standard syringe that can be purchased off-the-shelf, or it can be a custom syringe. Syringe 100 includes a syringe body 101, a pull rod 105 with a plunger fixedly coupled to a piston 103 disposed in the syringe body 101, and specifically within a lumen defined by an interior wall of the syringe body 101.

In some embodiments, the pull rod 105 can be configured for non-detachable engagement with the piston 103. Specifically, the pull rod 105 can be configured to be permanently coupled with the piston 103. This permanent coupling can be achieved via, for example, geometry of a tip of the pull rod 105 and/or of a receiving cavity within the piston 103. In some embodiments, for example, and as in the embodiment shown in FIG. 1C, a tip 120 of the pull rod 105 has a distal end 122 and a proximal end 124. The proximal end 124 of the tip 120 connects to a distal end 126 of a shaft 128. Together, the shaft 128 and the tip 120 form the pull rod 105.

In some embodiments, the distal end 122 of the tip 120 can shaped to facilitate insertion into a cavity of the piston 103, such that it becomes fixably coupled to the piston 103. This can include, for example the distal end 122 of the tip 120 include a taper 123, a filet, or the like. The proximal end 124 of the tip 120 can include one or several features to assist in retaining the coupling between the pull rod 105 and the piston 103. In some embodiments, this can include a shoulder 130 that can form an edge 132 extending partially or wholly around the proximal end 124 of the tip 120. In some embodiments, the shoulder 132 can extend radially from the pull rod 105, and can, in some embodiments, be perpendicular to the pull rod 105 or can form up to a 90 degree angle with respect to the pull rod 105 at the proximal end 124 of the tip 120.

The tip 120 is inserted into the piston 103 causing the piston 103 to be fixedly coupled to the pull rod 105. This pull rod 105 cannot be separated from the piston 103 once the pull rod 105 is engaged with the piston 103.

Typically, the piston 103 is disposed at a proximal end 110 of the standard syringe 100 when there is no fluid in the syringe 100. The piston 103 can be moved within the lumen defined by the syringe 100 to change a working volume of that syringe 100 and to thereby draw fluid into the syringe 100, expel fluid from the syringe 100, or hold fluid in the syringe 100.

As shown in FIG. 1B, the syringe 100 can aspirate a fluid LQ1 when the pull rod 105 is retracted, or in other words is pulled backward to move the piston 103 toward the distal end 112 of the syringe. A vacuum is created in the lumen of the syringe 100 between the piston 103 and the proximal end 110 of the syringe 100 when the piston 103 moves towards the distal end 112 of the syringe 100. This vacuum causes fluid LQ1 to be aspirated into the syringe 100. In the example shown, fluid LQ1 is drawn through a needle N1 coupled at the proximal end 110. In another example, a needle may not be used. Upon aspirating the fluid LQ1 into the syringe 100, a portion 105p of the pull rod 105 distally protrudes from the distal end 112 of the syringe 100.

The syringe 100 is not well suited for several applications because of the portion 105p of the pull rod 105 extending and exposed from the syringe 100. As such, a small amount of force on the pull rod 105 can undesirably cause syringe contents to exit the syringe 100 in an undesired manner, leading to loss of the contents into the system at a desired time and/or in an undesired quantity. For example, the rod portion 105p sticking out can be accidentally bumped, which can result in the inadvertent dispensing of the contents (e.g., the fluid LQ1, e.g., a liquid sample) at an unspecified time and/or of an unspecified amount.

The present disclosure describes a syringe that advantageously eliminates such unintentional dispensing by separating the piston from the pull rod. Specifically, described herein are syringes that include features enabling separation of the piston from a pull rod or plunger. For example, the separation/decoupling of the plunger can be achieved when the piston has been retracted. After separating the plunger, the piston remains in the syringe body, thereby retaining any contents (e.g., the fluid, a sample, etc.) within the syringe. The plunger can be subsequently re-engaged to dispense the contents when desired. The skilled artisan will appreciate that re-engagement of the plunger is not the only means by which the contents can be dispensed; rather, any means of depressing the piston can be used.

In one embodiment, for example, the pull rod can be configured to be removed from the piston. In one embodiment, an insert is provided to contact the piston so that the piston remains in the syringe body while the pull rod is decoupled from the piston and is removed from the syringe.

In some embodiments, the pull rod or the plunger can include a tip that is shaped and sized to allow easy detachment of the piston from the plunger. For example, the pull rod can include a tip having a smooth or rounded proximal end, as opposed a proximal end 124 having a shoulder 130 with sharp a sharp edge 132 as shown in FIG. 1C. Furthermore, in some embodiments, the tip can be sized to create a tight fit with the piston. The tight fit allows the piston to be pulled back and to be easily detached when desired, e.g., after the piston reaches the desired position in the syringe. In some embodiments, the tip of the plunger can have a curved (e.g., spherical) geometry so that it can be easily removed from the piston.

In some embodiments, an insert can be provided to stop the piston at a desired position and further allow the plunger to be removed from the syringe. For example, the insert can be a hollow insert, or barb, designed to be inserted into a syringe body at a desired location. In some embodiments, the insert can be made from a variety of materials, including, for example, stainless steel or polymer, such as plastic. The insert can have a variety of shapes and sizes, and can, in some embodiments, comprise a cylindrical insert defining a channel extending axially through the insert. The channel through the insert can be sized to allow the plunger to pass through the insert to engage with the piston, and in some embodiments, a diameter of the channel through the insert is larger than a diameter of the plunger. In some embodiments, the insert can protrude into the lumen of the syringe. The insert can thereby partially obstruct the lumen. The insert engages with the piston when the piston is retracted, and thereby retains the piston within the lumen and facilitates the separation of the plunger from the piston.

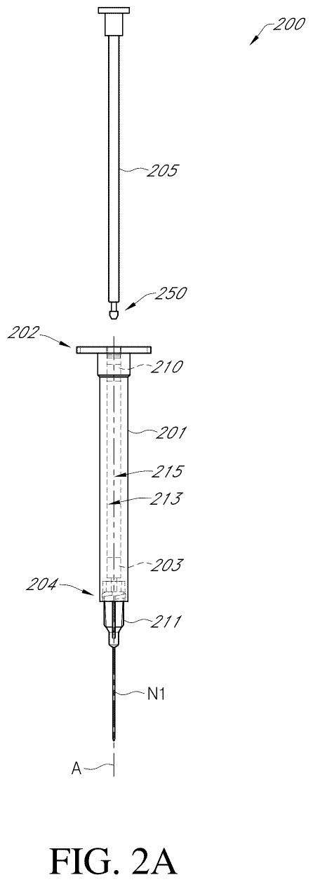

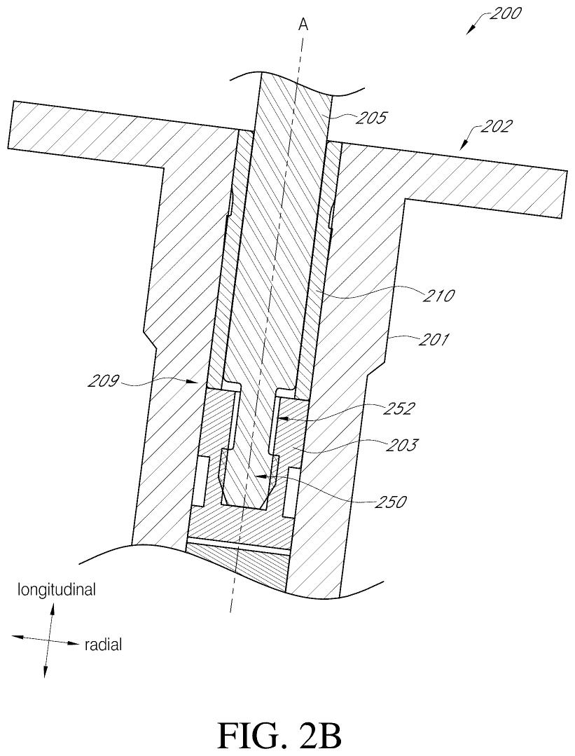

FIG. 2A illustrates one embodiment of an exemplary syringe 200 with a plunger 205 detached from the piston 203. FIG. 2B illustrates a cross section view of one embodiment of a plunger tip 250, engaged with a piston 203 of the syringe 200. The piston 203 can be moved within a lumen 215 of the syringe 200 to aspirate contents (e.g., a fluid, a sample, and the like) into the syringe 200 or to dispense contents (e.g., a fluid, a sample, and the like) from the syringe 200. The piston 203 can be moved by the plunger 205 when the plunger 205 is coupled to the piston 203. When the piston 203 is decoupled from the plunger 205, the syringe 200 can be empty or hold a desired amount of contents (e.g., a fluid, a sample, and the like). Advantageously, when the plunger 205 is detached from the syringe 200, the syringe 200 does not have any portion of plunger 205 distally protruding out of the syringe 200. As such, any accidental discharge of the contents (e.g., a fluid, a sample, and the like) from the syringe 200 can be prevented upon bumping on the plunger 205. The plunger 205 or another rod can be re-engaged with the piston 203 to discharge the contents (e.g., a fluid, a sample, and the like) from the syringe 200 when desired. Alternatively, the piston can be engaged by other means (e.g., an actuator or pressurized gas, pneumatic or otherwise) in order the facilitate discharge of the contents in the syringe.

As shown in FIG. 2A, the syringe 200 includes a syringe body 201 configured to hold contents (e.g., a fluid, a sample, and the like). The syringe body 200 includes an inner wall 213 defining a lumen 215. A piston 203 is movably disposed within the lumen 215 in the syringe body 201. In some embodiments, the piston 203 can be sized and configured to sealingly engage with the inner wall 213 such that desired contents such as a fluid can be aspirated into the lumen 215 when the piston 203 is distally retracted, and contents can be dispensed from the lumen 215 when the piston is proximally advanced. The syringe 200 can include a removable plunger 205 (also referred to as removable pull rod 205) that can be removably coupled to the piston 203. In some embodiments, the syringe 200 can include a stopper surface 209 to stop the piston 203 and to prevent the piston 203 from being removed from the syringe 200.

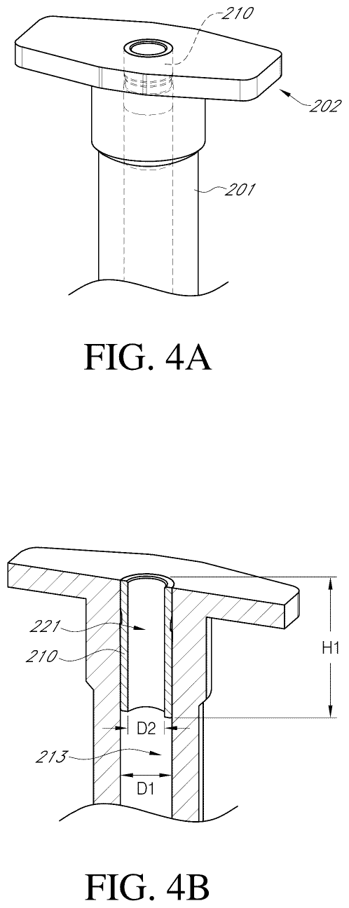

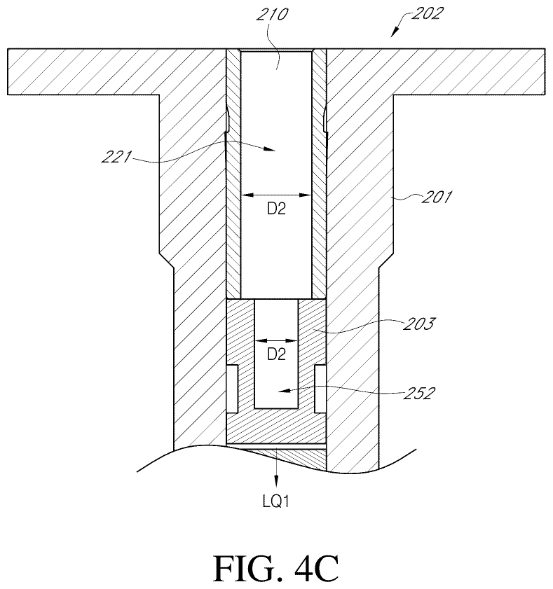

The syringe body 201 has an orifice, which can be a luer 211 fluidically connected to the lumen 215. As seen in FIG. 2A, the lumen 215 has a central longitudinal axis A, that can, in some embodiments, axially extend through the luer 211. The lumen 215 can further have a diameter D1 (see FIG. 4B).

Optionally, the luer 211 of the syringe 200 can be coupled to a needle N1, whereby contents (e.g., fluid, sample, and the like) can be aspirated into the lumen 215 or dispensed from the lumen 215. When present, needle N1 can be integral with the syringe 200, or detachable from the syringe 200.

In some embodiments, the syringe body 201 is made from material that enables visual detection of contents within the lumen, e.g., transparent plastic, transparent glass, and the like. The syringe body can be marked with a measurement scale to enable visualization of the amount of contents aspirated in the lumen 215. For example, the measurement scale (similar to shown in FIG. 1A) can be configured to measure a volume of contents aspirated in the lumen 215 in milliliters (ml), microliters (μl), ounces (oz), or other measurement units. In some embodiments, the syringe body 201 is composed from any known material typically used for a syringe such as a non-bioreactive plastic, glass or other material that does not react with the contents to be aspired in the syringe. In some embodiments, the syringe body 201 is composed of, or includes a bioreactive material that preserves or otherwise stabilizes the contents of the syringe to maintain extended longevity.

In some embodiments, the piston 203 is located in the lumen 215 of the syringe body 201. The piston 203 can be disposed axially in the lumen 215. The piston 203 comprises a top and a bottom, and sealing engages with the inner walls 213 of the syringe body 201. The bottom of the piston 203, together with the interior walls 213 of the syringe body 201 define a working volume configured to hold contents. The piston 203 is movable along the longitudinal axis A to change the working volume in the lumen 215. The piston 203 can be disposed at a proximal end 204 of the syringe 200 before receiving the fluid, and the piston 203 can be retracted by being pulled with the plunger 205 to a desired position in the lumen 215, which desired position can be at a distal end 202 of the syringe 200. The piston 203 can be retracted to fill the lumen 215 of the syringe 200 with contents.

The piston 203 and the plunger 205 are configured to be removably coupled to each other. For example, in the embodiment show in FIG. 2A, a cavity is provided in the top side of the piston 203. The cavity can be configured to engage or disengage with the plunger 205. In some embodiments, the cavity (e.g., see cavity 252 in FIGS. 2B and 4C) can extend partially toward the bottom of the piston 203. In some embodiments, the cavity does not extend through the bottom of the piston 203. The cavity can be formed at a center of the piston 203 and configured to mate with an extension from a distal end of the plunder 205. The cavity extends axially partially through the piston 203.

The removable plunger 205 is configured to removably couple with the piston 203. In some embodiments, a portion of the plunger 205 that engages with the piston 203 can be configured to easily couple and decouple from the piston 203. The portion of the plunger 205 is configured such that the plunger 205 stays coupled with the piston 203 when retracting the piston 203, but when retracted to the desired position, (e.g., when retracted such that the piston meets the stopper surface 209) the portion of the plunger 205 can be decoupled easily (e.g., simply pulling away the plunger 205 in an axial direction).

In some embodiments, the tip portion 250 of the removable plunger 205 can be configured to couple and decouple with the cavity of the piston 203. In some embodiments, the tip can be advanced into the cavity 252 of the piston 203. The tip 250 can be sized so as to deformably engage walls of the cavity 252 of the piston 203. This deformation and the thereby arising forces can couple the piston 203 to the tip 250 of the plunger 205. When coupled, the force can be applied to plunger 205 to longitudinally displace the plunger and to move the piston 203 inside the lumen 215 When the piston 203 reaches a desired position indicating the desired volume of fluid is filled in the syringe, the further distal movement of the piston 203 can be blocked by the insert 210, and the plunger 205 can separate from the piston 203, and specifically the tip 250 of the plunger 205 can be retracted from the cavity 252 of the piston 203. The tip portion 250 is tightly engaged with the cavity 252 of the piston 203 such that a force applied to displace the piston 203 is greater than a friction force between the piston 203 and the lumen 215, but less than a force required to decouple the plunger 205 from the piston 203.

In some embodiments, as illustrated in FIGS. 2B and 4C, the cavity 252 of the piston 203 can have a hollow cylindrical shape and sized to deformably receive the tip 250 and to provide a friction fit between a tip 250 of the removable plunger 205 and the piston 203. The cavity 252 can have an internal diameter D3 configured to receive the tip 250 of the plunger 205, which tip 250 has a larger diameter than the diameter D3 of the cavity 252. The cavity 252 can be sized to receive the tip 250 and tightly fit around the tip 250 such that the piston 203 is retractable by the removable plunger 205.

In some embodiments, the cavity 252 of the piston 203 can be sized smaller than a size of the tip 250 of the plunger 205. The shape of the cavity 252 can conform to a shape of the tip 250 of the removable plunger 205 such that when the piston 203 receives the removable plunger 205, the cavity 252 of the piston 203 expands around the tip 250 to couple the removable plunger 205 and the piston 203. It can be understood that the shape and size of the cavity 252 and the tip 250 are exemplary and does not limit the scope of the present disclosure. In some embodiments, the cavity 252 can have a different shape such as conical, rectangular, spherical, or other standard or custom geometric shapes, including combinations thereof that enables a fit between the cavity and the tip 250 of the plunger 205 such that the plunger 205 can be easily coupled and decoupled from the piston 203.

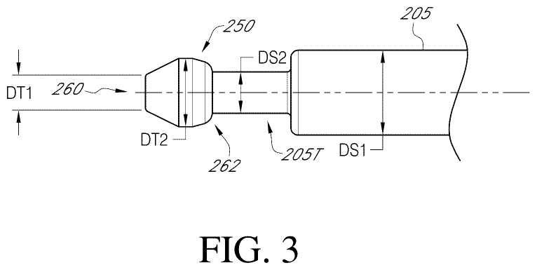

FIG. 3 illustrates an exemplary geometry of one embodiment of the plunger tip 250 that engages with the cavity 252 (shown in FIG. 2B) of the piston 203. Either end of tip 250 has sufficient draft or curvature such that the plunger 205 can engage and operate the piston 203 but become disengaged when sufficient opposing forces are applied between the plunger 205 and the piston 203. For example, as shown in FIG. 2B, the diameter of the tip 250 is larger than the diameter D3 of the cavity 252 (shown in FIG. 2B). In some embodiments, the tip 250 can be conical or spherical in shape. For example, the tip 250 can be conical in shape having a narrow proximal end 260, also referred to herein as narrow end 260 or proximal end 260, which can be configured to enter the cavity 252 in the piston 203 (shown in FIG. 2B) and a distal end 262, also referred to herein as base end 262, configured to couple the piston 203 to the tip 250. For example, the proximal end 260 has a diameter DT1, and the tip 250 can have a maximum diameter DT2 that is larger than the diameter DT1. The diameter DT1 of the narrow end 260 is smaller than the diameter D3 of the cavity 252 to allow at least partial insertion of the tip 250 without any resistance from the cavity 252 of the piston. The diameter DT2 is larger than the diameter D3 of the cavity 252 such that a force is required to push the tip 250 into the cavity 252 of the piston 203 to tightly engage the piston 203 and the plunger 205. Thus, the tip 250 facilitates coupling of the plunger 205 to the piston 203 such that when the piston is being retracted, the plunger 205 does not detach from the piston 203.

In some embodiments, the distal end 262 of the tip 250 has a reduced diameter as compared to diameter DT2. This reduced diameter can facilitate retraction of the tip 250 from the cavity 252 of the piston 203 and can thereby facilitate easy decoupling of the plunger 205 from the piston 203. In some embodiments, the reduced diameter can include a chamfered, spherical, or fillet edge extending around the distal end 262 of the tip 250 to create smooth edges. The smooth edges, and the shape and size of the tip 250 in cooperation with the cavity 252 enables the plunger 205 to be pushed into and pulled out of the piston with a small force. In some embodiments, the distal end 262 of the tip 250 does not include a shoulder having a sharp edge like that shown in FIG. 1C, and which is typically found on a typical plunger using in a typical syringe. Such sharp edges of the typical plunger dig into the piston when pulling out of the piston and prevent the plunger from being removed from the piston without damaging the piston. Instead, the distal end 262 comprises a reduced diameter that tapers towards the maximum diameter DT2. In some embodiments, the cavity 252 of the piston 203 can be shaped to conform to the base portion (e.g., portion at the distal end 262) of the tip 250 creating a fit between the plunger 205 and the piston 203 that assists with retraction of the piston 203 without decoupling from the plunger 205. For example, the cavity 252 can be cylindrically shaped, and have a diameter smaller than the reduced diameter of the distal end 262. In such embodiments, the cavity can deform around and conform to the shape of the tip 250, and specifically can deform around and conform to the shape of the distal end 262 of the tip 250.

In some embodiments, the removable plunger 205 can be a cylindrical rod having a shaft diameter DS1 with the tip 250 extending from a shaft portion 205T. The shaft portion 205T has a smaller diameter DS2 compared to the shaft diameter DS1 of the plunger 205. In some embodiments, the plunger 205 can have the shaft portion 205T with the diameter DS2 smaller than the size of the cavity 252 (e.g., the diameter D3), while the shaft diameter DS1 can be approximately equal to or larger than the size of the cavity 252 (e.g., diameter D3). The smaller diameter DS2 ensures that only the tip 250 engages with the cavity 252 of the piston 203 while the shaft portion 205T does not engage with the cavity 252. In some embodiments, the shaft diameter DS1 of the plunger 205 enables insertion of the tip 250 up to a maximum depth of the cavity 252 so that any forceful insertion into the cavity 252 does not cause the tip 250 to damage the piston 203, for example, the tip 250 tearing through the bottom end of the piston 203.

In some embodiments, the piston 203 can be made of compliant material e.g., elastic material such as a rubber material or silicone or other resiliently deformable material. The terms “elastic” and “elastic material” mainly refer to cross-linked thermoset polymers, such as silicone or rubber-like polymers, which are more easily deformable than plastics. Such material is, e.g., biologically and chemically inert/non-reactive and thus suitable for use with reactive or biological fluids and is not easily affected by leaching or gas movement under ambient temperature and pressure. In some embodiments, the plunger 205 can be made of metal (e.g., steel or the like), plastic or other materials harder than the piston 203. In some embodiments, the plunger 205 can have a surface roughness such that a friction fit is created with the material of the piston 203, but smooth enough to easily remove the plunger 205 from the piston 203.

In some embodiments, a stopper surface 209 is located at a desired position in the lumen 215 to prevent the piston 203 from retracting from the syringe body 201. The stopper surface 209 can, in some embodiments, narrow the lumen 215 and can be configured to prevent retraction of the piston 203 beyond the desired position. As an example, the stopper surface 209 can be a portion of an insert (e.g., 210 in FIG. 2A) fixedly attached at a desired position in the lumen 215 of the syringe 200. In some embodiments, the insert 210 is press-fit, glued, crimped or welded into position in the lumen 215. In some embodiments, the distal end 202 of the syringe 200 can be shaped to include one or more structural features, such as a protrusion, recess, groove or slot to prevent movement of the stopper surface 209 beyond the desired point in the syringe 200. FIG. 4A shows a perspective view of one embodiment of the insert 210 (including stopper surface 209 as depicted in FIG. 2B) in the syringe 200 and FIG. 4B shows a cross section view of one embodiment of the insert 210 in the syringe 200. FIG. 4C is a cross section view of one embodiment of the insert 210 engaged with the piston 203 of the syringe 200 so that the plunger 205 can be decoupled from the piston 203.

As shown in FIGS. 4A-4C, the insert 210 can comprise a hollow cylinder defining an internal channel 221. The internal channel 221 has a diameter that is larger than an outside diameter (e.g., D1) of the removable plunger 205. In some embodiments, the internal channel 221 can be configured to allow the removable plunger 205 to movably extend through the internal channel 221 and move along the longitudinal axis A, and to remove the removable plunger 205 from the syringe 200 when decoupled from the piston 203. In some embodiments, the insert 210 can be inserted (e.g., removably inserted, or permanently inserted) at the distal end 202 of the syringe 200 opposite the luer 211. In some embodiments, the insert 210 can be fixedly attached at the distal end 202 of the syringe at the desired position in the lumen 215. In some embodiments, the insert 210 can be a ring integrally formed inside the syringe 200 or projections extending radially inward. For example, a hot metal rod, ultrasonic welder, or electromagnetic radiation, e.g., laser, can be used to melt portions (e.g., internal and/or external portions) of the syringe body 201 to form a ring, flange, ledge or other structure that acts as an insert having a stopper surface. Accordingly, instead of adding a part into the syringe 200, the existing syringe plastic can be melted or otherwise altered to form the insert 210 having the stopper surface 209.

As shown in FIG. 4C, the internal channel 221 of the insert 210 can have a diameter D2, which is larger than the diameter D3 of the cavity 252, and smaller than an outer diameter of the piston 203. The insert 210 has a length H1 such that a bottom end extends up to a desired position at which the piston 203 can be retracted and a top end that can be, in some embodiments, approximately flush with the distal end 202 of the syringe 200. As the internal channel 221 is smaller in size than the outer diameter of the piston 203, the insert 210 blocks the piston 203 and prevents the piston 203 from being completely removed from the distal end 202 of the syringe 200 when aspirating contents into the syringe. Also, while the insert 210 restricts the piston 203, the removable plunger 205 can be pulled out of the cavity 252 by applying a small amount of pulling force to decouple from the piston 203. Upon decoupling, the plunger 205 can freely pass through the internal channel 221 and can be completely removed from distal end 202 of the syringe 200.

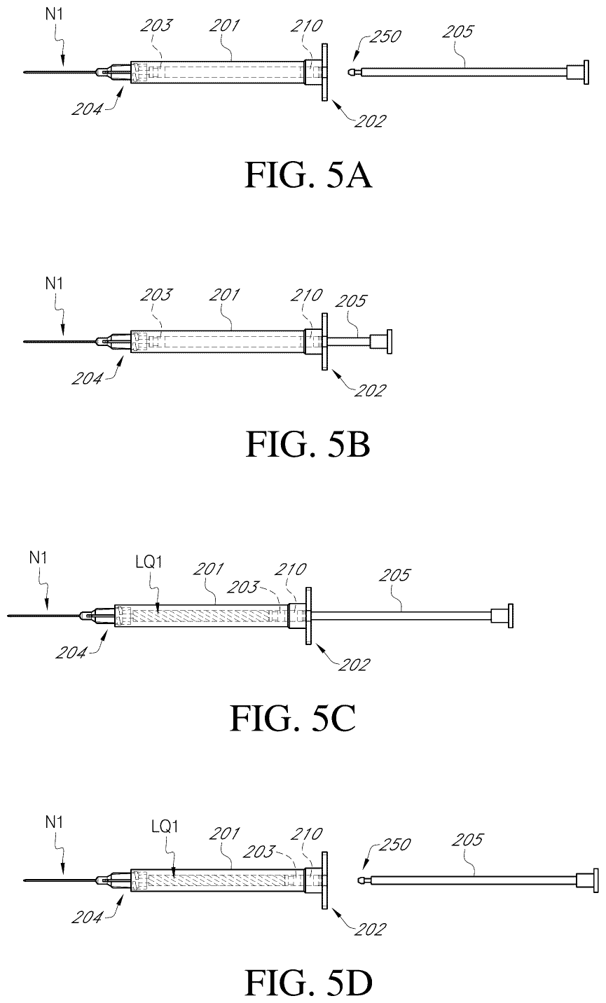

FIGS. 5A-5D illustrate one embodiment of a method of aspirating fluid into a syringe and removing the plunger from the syringe. In some embodiments, a syringe 200 comprising a piston 203, a removable plunger 205, and an insert 210 can be received, as shown in FIG. 5A. In some embodiments, the syringe 200 can be optionally coupled to the needle N1. The removable plunger 205 can be inserted from a distal end 202 of the syringe 200 into a lumen 215 defined by interior walls 213 of the syringe 200 to couple with the piston 203, as shown in FIG. 5B. In some embodiments, inserting the removable plunger 205 involves aligning the tip 250 of the removable plunger 205 with the cavity 252 of the piston 203, and pushing the tip 250 into the cavity 252 until the removable plunger 205 is coupled with the piston 203. As discussed herein, the tip 250 is configured to couple with the cavity 252 of the piston 203 to move the piston inside syringe body 201 when a force is applied at the removable plunger 205 and the plunger 205 is longitudinally displaced. The tip 250 of the plunger is further configured to decouple from the piston 203 when the piston 203 engages with the insert 210.

The removable plunger 205 can be retracted distally to retract the piston 203 from a proximal end 204 of the syringe 200 toward the distal end causing a fluid LQ1 to enter the syringe 200 via a luer 211 operably connect with the needle N1 as shown in FIG. 5C. Also, as shown in FIG. 5C, further distal retraction of the piston can be prevented via the insert 210 positioned within the lumen 215 of the syringe body 201. As the piston 203 is blocked by the insert 210, further retraction of the plunger 205 can decouple the plunger 205 from the piston 203, and thus the removable plunger 205 can be removed from the piston 203, as shown in FIG. 5D.

In some embodiments, removing the removable plunger 205 involves further retracting the removable plunger 205 while the piston 203 is engaged with the insert 210, thereby removing the tip 250 of the removable plunger from the cavity 252 of the piston 203 such that the piston 203 stays inserted in the syringe, while the removable plunger 205 is completely detached from the piston 203.

As discussed herein, the insert 210 can be fixed in the syringe body 201 or removably inserted from the distal end 202 of the syringe 200. Accordingly, the method can further include securing the insert 210 at a desired position in the lumen of the syringe body 201, where the desired position corresponds to an amount of contents (e.g., a fluid, a sample, and the like) to be filled in the syringe.

Once the desired amount of contents (e.g., a fluid, a sample, and the like) is aspirated into the syringe 200, the loaded syringe 200 can be used in different applications such as preparing for a gene editing step of CAR T-cell therapy. Accordingly, it will be appreciated that depending upon the particular application, multiple syringes 200 can be filled with different types of contents, such as biomolecules (e.g., Cas9 protein, guide RNA, and donor DNA, respectively for gene editing applications). The syringes 200 can then be loaded into a receptacle configured to deliver the contents to a system. Advantageously, as the syringes 200 have no plunger 205 portion sticking out of the syringe body 201, the syringes 200 can be easily transported and handled without accidentally discharging or mixing of fluids.

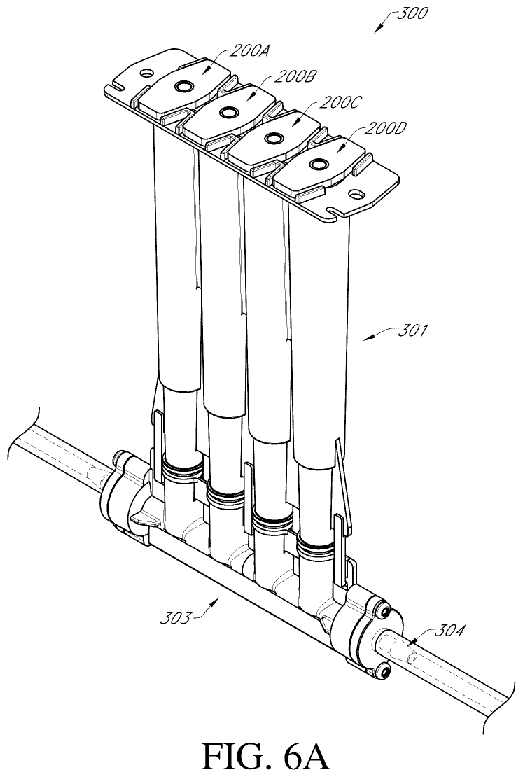

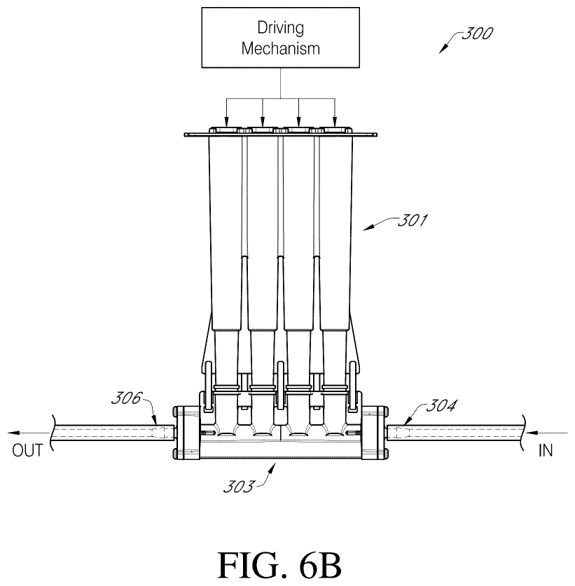

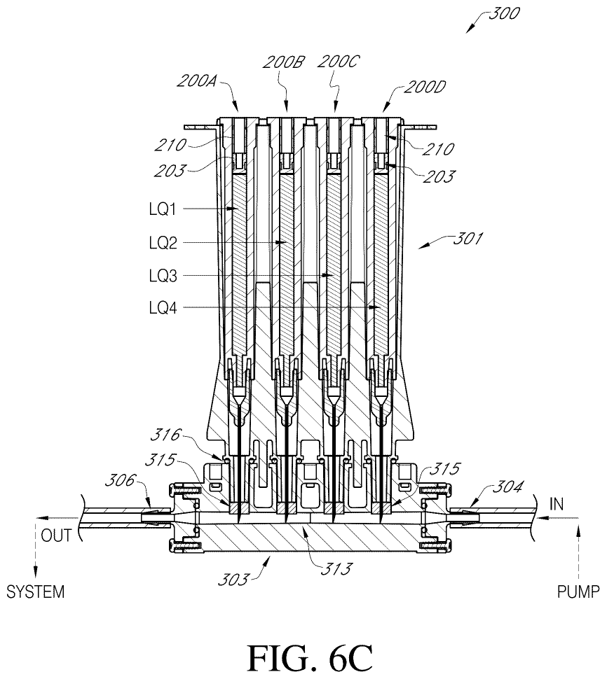

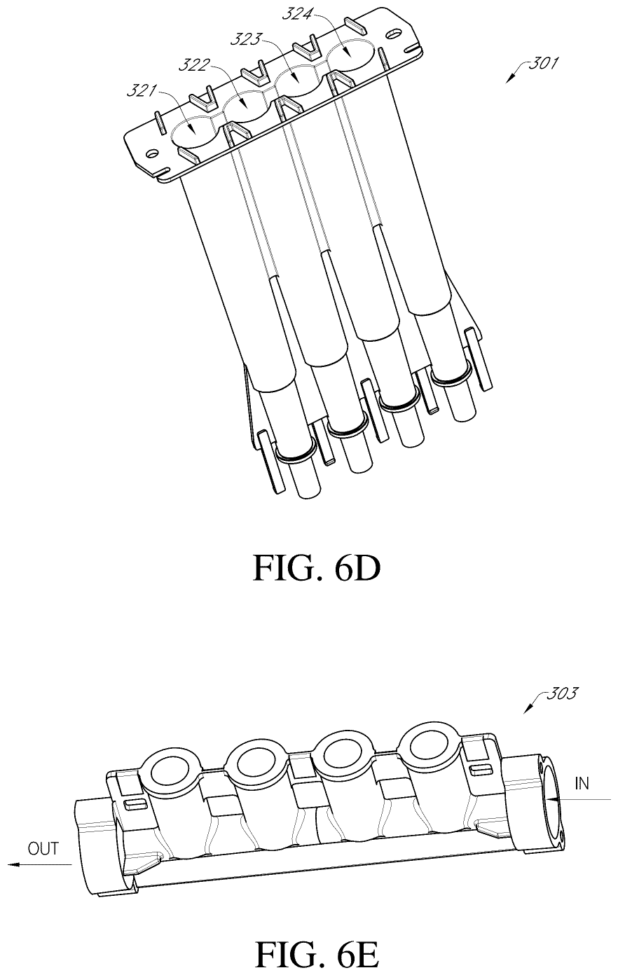

FIG. 6A illustrates an exemplary system 300 employing multiple syringes 200 (discussed with respect to FIGS. 2-5) in a receptacle 301, according to some embodiments. The receptacle 301 is configured to receive one or several syringes 200, and specifically, the embodiment shown in FIGS. 6A and 6D is configured to receive a plurality of syringes such as 200A, 200B, 200C, and 200D that carry same or different type of contents (e.g., LQ1, LQ2, LQ3, LQ4, or other samples) in same or different amounts. In some embodiments, the receptacle 301 can, together with other components, be configured to inject fluid from one or more syringes 200A-200D at a desired time and at a desired amount.

FIG. 6B is a front view of one embodiment of the receptacle 301 and FIG. 6C is a cross section view of one embodiment of the receptacle 301. In some embodiments, the receptacle 301 (see also FIG. 6D) can be coupled to a feedline 303 (see also FIG. 6E) between an inlet 304 and an outlet 306. In operation, the inlet 304 can receive a sterilized gas or air (e.g., filtered or sterilized), or a liquid carrier (e.g., a buffer, water, or the like) from a pump. Contents (e.g., fluids LQ1-LQ4) can be injected into the receptacle 301 via syringes 200A, 200B, 200C, 200D, where the contents of the syringes are displaced by the fluid delivered, via the outlet 306. For example, in some embodiments, the outlet 306 is connected (e.g., in a closed, sterile manner), to a system such as an electroporation or other transfection system (e.g., as described in International Patent Application No. PCT/US2020/05713, which is incorporated by reference herein in its entirety), or culture system. As such, the systems described herein may be used for generating a cell therapy or treatment using genetically modified cells, CAR T cells, NK cells, stem cells, and the like.

Referring to FIGS. 6A-6C, the system 300 includes a syringe such as the syringe 200A, a feedline 303, and a syringe receptacle 301 configured to receive the syringe and fluidically couple the syringe to the feedline 303. The feedline 303 can be configured to deliver droplets from the syringe (e.g., 200A, 200B, 200C, and/or 200D) for developing a solution for a test or a therapy. The feedline 303 can include a channel 313 to carry fluid between the inlet 304 and the outlet 306.

As discussed herein, the syringe 200A (or 200B-200D) includes the syringe body 201 having interior walls defining a lumen 215 having a longitudinal axis, a removable plunger 205 including the tip 250, the piston 203 located in the lumen 215 of the syringe body 201, and the insert 210 narrowing the lumen 215. The piston 203 together with the interior walls 213 define a working volume configured to hold fluid. The piston 203 includes the cavity 252 configured to engage with the tip 250 of the removable pull rod 205. The piston 203 is movable along the longitudinal axis to change the working volume. The insert 210 narrows the lumen to prevent retraction of the piston 203 beyond the desired position.

In some embodiments, the receptacle 301 and the syringe 200A are coupled. In some embodiments, a portion of the syringe 200 can engage with a portion of the receptacle 301 to couple the syringe 200 and the receptacle 301. In some embodiments, the receptacle 301 and the syringe 200A are coupled by a fastening mechanism, such as a snap, latch or threaded engagement. For example, in one embodiment, the fastening mechanism is a threaded engagement configured as a screw-type mechanism including threads formed at a proximal end on an outer surface of the syringe 200A that are received by threads formed on the receptacle 301 at an interior portion of the receptacle 301. Additional syringes (e.g., 200B, 200C and 200D) can be coupled to the receptacle in the same manner as the syringe 200A. In other embodiments, and as shown in FIG. 6C, the one or more syringes (e.g., 200A-200D) can additionally, or alternatively, be coupled to the receptacle 401 via the needle (e.g., N1) penetrating the septum 315 (e.g., made of a pierceable material, such as elastomeric material, rubber, silicone, cork, and the like).

In some embodiments, the receptacle 301 can be further sealed to the feedline 303 by one or several O-rings, gaskets, or other sealing means. For example, as shown in FIG. 6C, the receptacle 301 can be inserted in the feedline 303 and an O-ring 316 can be positioned proximate the inserted portion of the receptacle and proximate mating surfaces of both the receptacle 301 and the feedline 303. In some embodiments, these O-rings 316 also prevent ingress of any material from the outside around the O-ring portion. This also creates a secondary seal at the bottom part of the receptacle 301.

In some embodiments, the feedline 303 and the receptacle 301 can be coupled together. In some embodiments, the receptacle 301 and the feedline 303 can be coupled together via one or several fastening members such as one or several screws, bolts, nuts, or the like, or glued together. In some embodiments, the receptacle 301 and the feedline 303 can be coupled together via one or several mating features, which can snap together and/or matingly engage to secure and/or releasably secure the receptacle 301 to the feedline 303.

As illustrated in FIG. 6C and 6D, the receptacle 301 includes a plurality of hollow chambers 321-324 (marked in FIG. 6D), each chamber can be configured to receive one syringe. For example, a first hollow chamber 321 receives the syringe 200A, a second hollow chamber 322 receives the syringe 200B, a third hollow chamber 323 receives the syringe 200C, and the fourth hollow chamber 324 receives the syringe 200D. In some embodiments, one or more hollow chambers of the receptacle are configured to receive one or more syringes of different sizes. The plurality of chambers 321-324 are isolated from each other. In some embodiments, the receptacle 301 can be made of plastic, or other material.

In some embodiments, as shown in FIG. 6C, the receptacle 301 includes a septum 315 to be penetrated by a needle N1 attached at a proximal end of the syringe such that a tip of the needle enters the feedline to supply droplets into the feedline. The septum 315 can be rubber or other material capable of being pierced while maintaining a closed, sterile, fluid path. The syringe 200A is axially aligned with the septum 315. In some embodiments, the septum 315 is installed close to a top of the feedline 303 (e.g., at a top of the channel 313) to prevent leakage of the fluid or gas from the feedline and to create a closed, sterile fluid connection 303 into the receptacle 301. In some embodiments, the septum 315 can enter the feedline 303 to ensure a tight seal.

In some embodiments, as shown in FIGS. 6B, 6C and 6E, the feedline 303 has the inlet 304 and the outlet 306. The receptacle 301 is connected between the inlet 304 and the outlet 306 of the feedline 303. In some embodiments, the inlet 304 can receive sterilized gas or air from a pump and the outlet 306 can deliver a mixture of contents from the plurality of syringes 200A-200D in the receptacle 301.

In some embodiments, a drive mechanism (see FIG. 6B) can be configured to inject, at a specified time and a specified amount, contents from one or more syringes 200A-200D into the feedline 303. When the system 300 determines contents are to be injected, an individual piston 203 will be driven downward until it has a desired amount of contents is injected into the feedline 303 or until the piston 203 has bottomed. The contents from the syringe e.g., 200A will enter the feedline 303 and will be driven to its destination by sterile air or buffer supplied at the inlet 304, or vacuum applied at an outlet 306.

In some embodiments, once a piston 203 of a syringe, e.g., 200A, has been driven downward, it will be held in the downward position until all other syringes 200B, 200C, and 200D have been dispensed. This prevents subsequent contents from exiting the feedline 303 and entering an empty syringe.

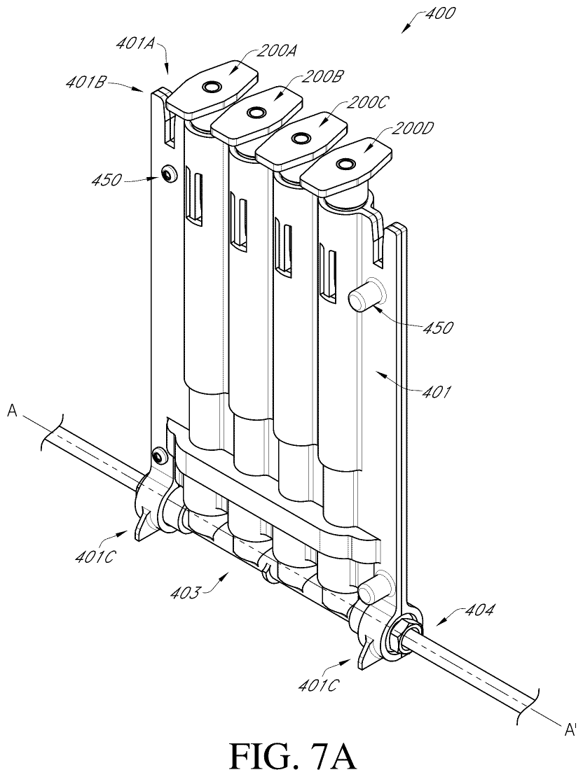

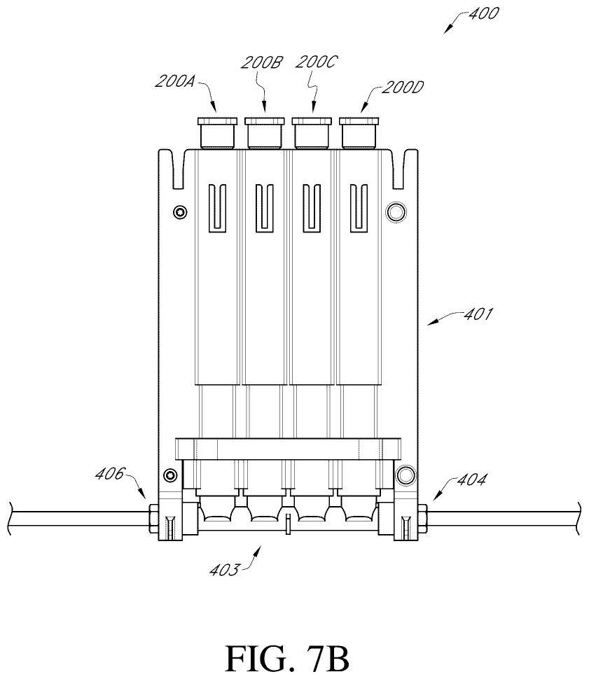

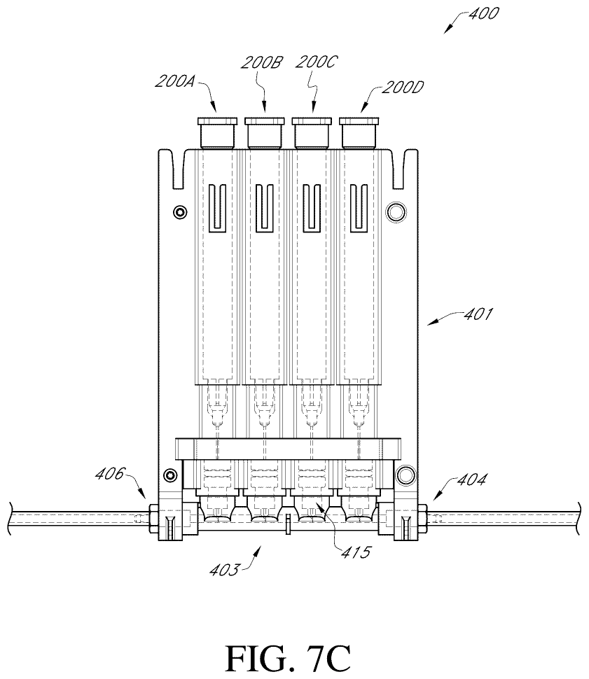

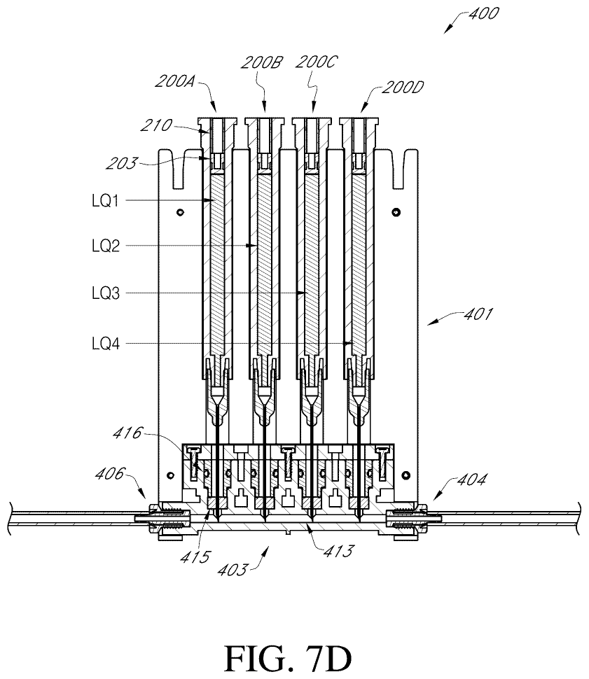

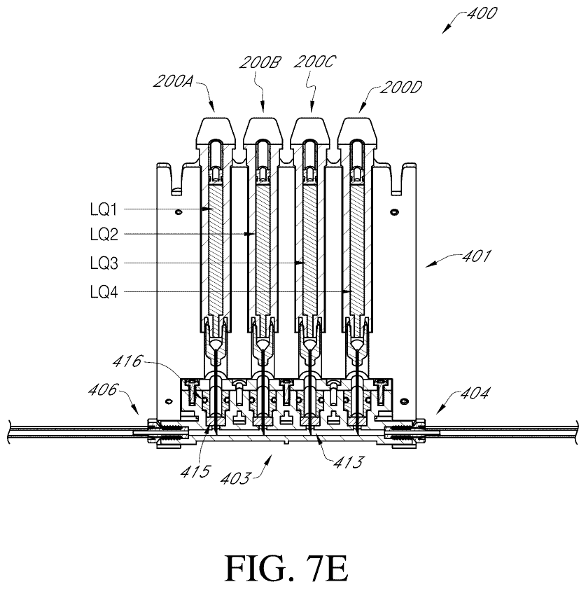

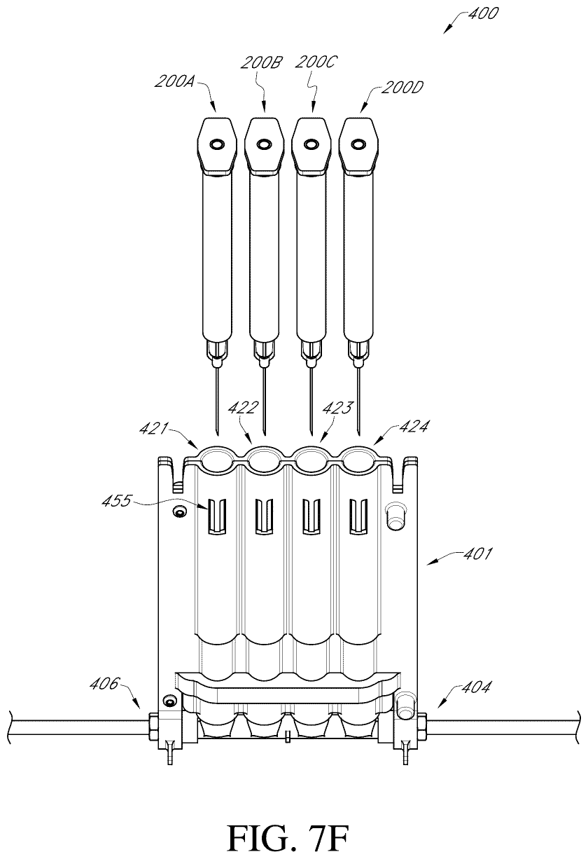

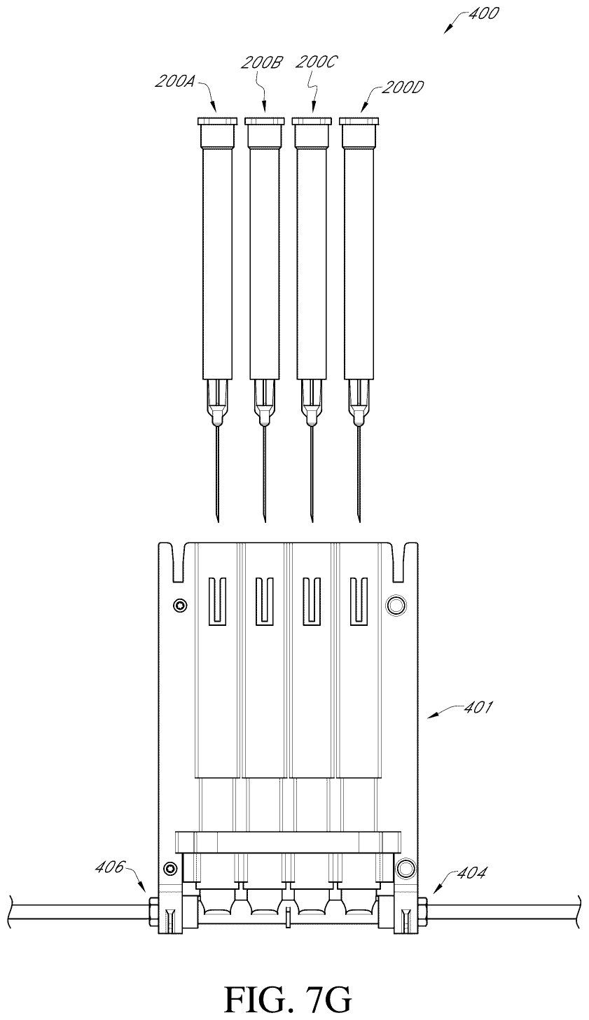

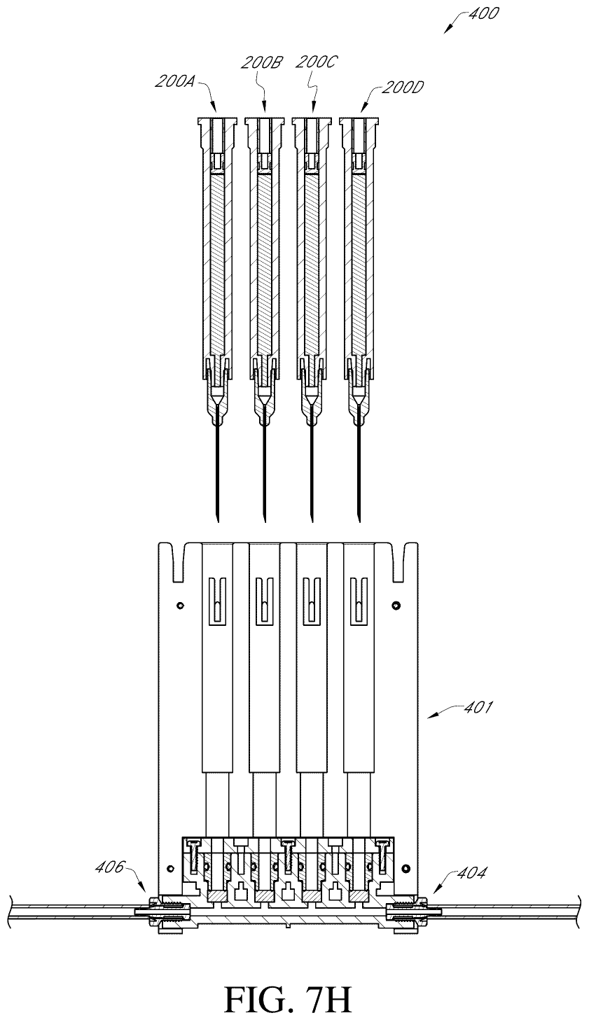

FIGS. 7A-7H illustrate an exemplary system 400 employing multiple syringes 200 (discussed with respect to FIGS. 2-5) in a receptacle 401, according to some embodiments. The receptacle 401 is configured to receive several syringes 200, such as 200A, 200B, 200C, and 200D that carry the same or different type of contents (e.g., LQ1, LQ2, LQ3, LQ4, or other samples) in the same or different amounts. In some embodiments, the receptacle 401 can, together with other components, be configured to inject fluid from one or more syringes 200A-200D at a desired time and at a desired amount.

It will be appreciated that while FIGS. 7A-7H illustrate an embodiment of the disclosure including 4 syringes, the system 400 can be configured to include more or less than 4 syringes. For example, in various embodiments, system 400 can be configured to include 1, 2, 3, 4, 5, 6, 7, 8, 9, 10 or more syringes by expanding or contracting the configuration depicted in FIGS. 7A-7I, such as by increasing or decreasing the number of syringes and associated components.

FIGS. 7B and 7C are front views of one embodiment of system 400 and FIG. 7D is a cross section view of one embodiment of system 400. In some embodiments, the receptacle 401 can be coupled to a feedline 403 between an inlet 404 and an outlet 406. In operation, the inlet 404 receives a sterilized gas or air (e.g., filtered or sterilized air), and/or a liquid carrier (e.g., a buffer, water, or the like) from a pump. Contents (e.g., fluids LQ1-LQ4) can be injected into the receptacle 401 via syringes 200A, 200B, 200C, 200D, where the contents of the syringes are displaced by the air or the carrier delivered, via the outlet 406. For example, in some embodiments, the outlet 406 is connected (e.g., in a closed, sterile manner), to a system such as an electroporation or other transfection system (e.g., as described in International Patent Application No. PCT/US2020/05713, which is incorporated by reference herein in its entirety), or culture system. As such, the systems described herein for generating a therapy or treatment such as in CAR T-cell therapy.

Referring to FIGS. 7A-7H, the system 400 includes a syringe such as the syringe 200A, a feedline 403, and a syringe receptacle 401 configured to receive the syringe and fluidically couple the syringe to the feedline 403. The feedline 403 can be configured to deliver droplets from the syringe (e.g., 200A, 200B, 200C, and/or 200D) for developing a solution used in performing an assay. The feedline 403 can include a channel 413 to carry fluid between the inlet 404 and the outlet 406.

As discussed herein, the syringe 200A (or 200B-200D) includes the syringe body 201 having interior walls defining a lumen 215 having a longitudinal axis, a removable plunger 205 including the tip 250, the piston 203 located in the lumen 215 of the syringe body 201, and the insert 210 narrowing the lumen 215. The piston 203 together with the interior walls 213 define a working volume configured to hold fluid. The piston 203 includes the cavity 252 configured to engage with the tip 250 of the removable pull rod 205. The piston 203 is movable along the longitudinal axis to change the working volume. The insert 210 narrows the lumen to prevent retraction of the piston 203 beyond the desired position.

In some embodiments, the receptacle 401 and the syringe 200A are coupled. In some embodiments, a portion of the syringe 200 can engage with a portion of the receptacle 401 to couple the syringe 200 and the receptacle 401. In some embodiments, the receptacle 401 and the syringe 200A are coupled by a fastening mechanism. For example, in one embodiment, one or more hollow chambers 421-424 (marked in FIG. 7F) include a latch structure 455 configured to engage a structure formed on the syringe body 201 to retain the syringe 200A within the receptacle 401. Alternatively, the fastening mechanism can be configured as a screw-type mechanism including threads formed at a proximal end on an outer surface of the syringe 200A that are received by threads formed on the receptacle 401 at an interior portion of the receptacle 401. Additional syringes (e.g., 200B, 200C and 200D) can be coupled to the receptacle in the same manner as the syringe 200A. In other embodiments, and as shown in FIGS. 7C and 7D, the one or more syringes (e.g., 200A-200D) can additionally, or alternatively, be coupled to the receptacle 401 via the needle (e.g., N1) penetrating the septum 415 (e.g., made of a pierceable material, such as elastomeric material, rubber, silicone, cork, and the like).

In some embodiments, the receptacle 401 can be further sealed to the feedline 403 by one or several O-rings, gaskets, or other sealing means. For example, as shown in FIGS. 7C and 7D, the receptacle 401 can be inserted in the feedline 403 and an O-ring 416 can be positioned proximate the inserted portion of the receptacle and proximate mating surfaces of both the receptacle 401 and the feedline 403. In some embodiments, these O-rings 416 also prevent ingress of any material from the outside around the O-ring portion. This also creates a secondary seal at the bottom part of the receptacle 401.

In some embodiments, the receptacle 401 is formed as a unitary structure that is configured to couple with the feedline 403. In some embodiments, the receptacle 401 is formed as a unitary structure that is configured to couple with the feedline 403, in which the receptacle 401 includes a front portion 401A and a back portion 401B connected by a hinge region 401C, wherein the front portion 401A and the back portion 401B are coupled to form the receptacle 401. In the embodiment shown in FIG. 7A, the receptacle 401 includes front portion 401A and back portion 401B that are mechanically coupled together by one or more threaded connections 450 including screws. As further illustrated in FIG. 7A, the front portion 401A and the back portion 401B are connected by hinge portion 401C and have a clam-shell configuration in which the receptacle 401 is formed by bringing the front portion 401A and the back portion 401B into contact about a longitudinal axis A-A′ of the hinge portion 401C and coupling the front portion 401A and the back portion 401B together.

In some embodiments, the receptacle 401 is formed from discrete components and configured to couple with the feedline 403. For example, in one embodiment, the receptacle 401 is formed by coupling the front portion 401A and the back portion 401B, wherein the front portion 401A and the back portion 401B are formed as discrete components before assembly of receptacle 401.

It will be appreciated that while the front and back portions of the receptacle 401 depicted in FIG. 7A are coupled using one or more threaded connections including screws, any suitable type of fastening connection may be utilized, such as, for example, one or more press-fit connections, latches, snaps, chemical or thermal bonds or welds, adhesive or the like.

In various embodiments, the feedline 403 and the receptacle 401 are configured to be coupled together. In some embodiments, the receptacle 401 and the feedline 403 can be coupled via one or more fastening connections, such as one or several screws, bolts, nuts, press-fit connections, latches, snaps, chemical or thermal bonds or welds, adhesive or the like. In some embodiments, the receptacle 401 and the feedline 403 can be coupled together via one or several mating features, which can snap together and/or matingly engage to secure and/or releasably secure the receptacle 401 to the feedline 403. In some embodiments, the feedline 403 and the receptacle 401 are coupled together before receptacle 401 is fully formed by bringing the front portion 401A and the back portion 401B into contact about a longitudinal axis of the hinge portion 401C and coupling the front portion 401A to the back portion 401B.

As illustrated in FIGS. 7C and 7D, in some embodiments the receptacle 401 includes a plurality of hollow chambers 421-424 (marked in FIG. 7F), each chamber being configured to receive one syringe. For example, a first hollow chamber 421 receives the syringe 200A, a second hollow chamber 422 receives the syringe 200B, a third hollow chamber 423 receives the syringe 200C, and the fourth hollow chamber 424 receives the syringe 200D. In some embodiments, one or more hollow chambers of the receptacle 401 are configured to receive one or more syringes of different sizes. In some embodiments, the plurality of chambers 421-424 are isolated from each other. In some embodiments, the receptacle 401 is composed of a polymeric material, such as plastic.

In some embodiments, as shown in FIGS. 7C-7E, the receptacle 401 includes a septum 415 to be penetrated by a needle N1 attached at a proximal end of the syringe such that a tip of the needle enters the feedline to supply droplets into the feedline. In embodiments, the septum 415 is composed of silicone, elastomeric material, rubber or other material capable of being pierced while maintaining a closed, sterile, fluid path. The syringe 200A is axially aligned with the septum 415. In some embodiments, the septum 415 is installed close to a top of the feedline 403 (e.g., at a top of the channel 413) to prevent leakage of the fluid or gas from the feedline and to create a closed, sterile fluid connection 403 into the receptacle 401. In some embodiments, the septum 415 can enter the feedline 403 to ensure a tight seal.

In some embodiments, as shown in FIGS. 7C-7H, the feedline 403 has the inlet 404 and the outlet 406. The receptacle 401 is connected between the inlet 404 and the outlet 406 of the feedline 403. In some embodiments, the inlet 404 can receive sterilized gas or air from a pump and the outlet 406 can deliver a mixture of contents from the plurality of syringes 200A-200D into the receptacle 401.

In embodiments, a drive mechanism can be configured to inject, at a specified time and a specified amount, contents from one or more syringes 200A-200D into the feedline 403. When the system 400 determines contents are to be injected, an individual piston 203 will be driven downward until it has a desired amount of contents is injected into the feedline 403 or until the piston 203 has bottomed. The contents from the syringe e.g., 200A will enter the feedline 403 and will be driven to its destination by a fluid supplied at the inlet 404.

In some embodiments, once a piston 203 of a syringe, e.g., 200A, has been driven downward, it will be held in the downward position until all other syringes 200B, 200C, and 200D have been dispensed. This prevents subsequent contents from exiting the feedline 403 and entering an empty syringe.

In various embodiments, the driving mechanism for advancing the piston 203 downward can be a pneumatic mechanism, a lead screw mechanism, a loaded spring mechanism, or other driving mechanisms. For example, the pneumatic mechanism can be include creating a face seal with the top of each syringe 200A-200D and pressurizing the syringe internals to drive the piston downward. In some embodiments, a vision system or hall-effect sensor can be required to determine piston 203 location and to generate feedback indicative of an amount of contents dispensed in the feedline 303 or 403.

In various embodiments, a lead screw mechanism used in the system of the disclosure includes a linear actuator, such as a leadscrew and stepper motor configured to drive each piston downward. Limit switches and encoders can be incorporated to determine whether the pistons are in the up/down position.

In various embodiments, a loaded spring mechanism used in the stem of the disclosure includes one or several compression springs configured to compress upon removing the syringe and configured to thereby store energy for the next run. When a syringe is received, each spring can be individually released to drive a piston. The release of the spring can be achieved by a mechanical actuator, including for example, one or more solenoids and/or cam mechanisms. In some embodiments, dampened springs can be used to make the spring slower to deploy and less aggressive. Mechanical limit switches can be used to determine up/down positions of the piston.

While certain embodiments have been described, these embodiments have been presented by way of example only, and are not intended to limit the scope of the present disclosures. Indeed, the novel methods, apparatuses and systems described herein can be embodied in a variety of other forms; furthermore, various omissions, substitutions and changes in the form of the methods, apparatuses and systems described herein can be made without departing from the spirit of the present disclosures. The accompanying claims and their equivalents are intended to cover such forms or modifications as would fall within the scope and spirit of the present disclosures.

What is claimed is:

1. A syringe comprising:
a syringe body having an inlet and interior walls defining a lumen having a longitudinal axis and a diameter;
a piston located in the lumen of the syringe body, the piston having a top and a bottom, the bottom of the piston together with the interior walls defining a working volume, the piston comprising a cavity, wherein the piston is movable along the longitudinal axis to change the working volume;
an insert located at a desired position in the lumen, the insert narrowing the lumen and being configured to prevent retraction of the piston beyond the desired position; and
a removable pull rod configured to removably couple to the piston, the removable pull rod comprising a tip configured to couple with the cavity of the piston and move the piston inside syringe body when a force is applied at the removable pull rod, and to allow decoupling from the piston when the piston engages the insert at the desired position.

2. The syringe of claim 1, wherein the tip is conical in shape having a narrow end configured to enter the cavity in the piston and a base end configured to couple the piston to the tip.

3. The syringe of claim 2, wherein the base end of the tip has a chamfered, spherical, or fillet edge configured to facilitate retraction of the tip from the cavity of the piston.

4. The syringe of claim 2, wherein the base end of the tip does not include a sharp edge having a 90 degree angle between adjacent surfaces.

5. The syringe of claim 2, wherein the piston is made of a compliant material.

6. The syringe of claim 5, wherein the cavity is cylindrical in shape and sized to provide a friction fit between the tip of the removable pull rod and the piston.

7. The syringe of claim 5, wherein the cavity is sized to receive the tip and tightly fit around the tip such that the piston is retractable by the removable pull rod.

8. The syringe of claim 2, wherein the base end comprises a base portion configured to taper away from the narrow end.

9. The syringe of claim 8, wherein the cavity of the piston is configured to cover the base portion of the tip.

10. The syringe of claim 9, wherein the piston is made of a compliant material, wherein the cavity of the piston sized smaller than a size of the tip and is configured to conform to a shape of the tip of the removable pull rod, wherein when the piston receives the removable pull rod, the cavity of the piston expands around the tip thereby coupling the removable pull rod and the piston.

11. The syringe of claim 1, wherein the insert comprises a hollow cylinder defining an internal channel, wherein the internal channel is larger than an outside diameter of the removable pull rod and is configured to allow the removable pull rod to movably extend through the internal channel and move along the longitudinal axis, and to remove the removable pull rod from the syringe when decoupled from the piston.

12. The syringe of claim 1, wherein the insert is removably inserted at a distal end of the syringe opposite the inlet.

13. The syringe of claim 1, wherein the insert is fixedly attached at a distal end of the syringe at the desired position in the lumen.

14. The syringe of claim 1, wherein the piston is disposed at a proximal end of the syringe configured to receive the fluid, and the desired position is at a distal end of the syringe indicative of a desired amount of the fluid to be filled in the syringe.

15. The syringe of claim 14, wherein the cavity extends partially toward the bottom of the piston but does not extend through the bottom of the piston.

16. The syringe of claim 1, wherein the piston is disposed axially in the syringe body, and the cavity is formed at a center of the piston and extends axially.

17. A method of aspirating a syringe, the syringe comprising a piston and a removable pull rod, the method comprising:
inserting the removable pull rod from a distal end of the syringe into a lumen defined by interior walls of the syringe to couple with the piston;
retracting the removable pull rod to retract the piston from a proximal end of the lumen toward the distal end causing a fluid to enter the syringe via an inlet in the proximal end;
preventing further retraction of the piston via an insert positioned within the lumen; and
removing the removable pull rod from the piston.

18. The method of claim 17, wherein inserting the removable pull rod comprises:
aligning a tip of the removable pull rod with a cavity of the piston; and
pushing the tip into the cavity until the removable pull rod is coupled with the piston,
wherein the tip is configured to couple with the cavity of the piston to move inside syringe body when a force is applied at the removable pull rod, and is configured to decouple from the piston when the piston engages with the insert.

19. The method of claim 18, wherein removing the removable pull rod comprises further retracting the removable pull rod while the piston is engaged with the insert, thereby removing the tip of the removable pull rod from the cavity of the piston such that the piston stays inserted in the syringe, while the removable pull rod is completely detached from the piston.

20. The method of claim 17, further comprising:
securing the insert at a desired position in the lumen, the desired position corresponding to an amount of fluid to be filled in the syringe.

21. A system comprising:
a syringe, comprising:
a syringe body having interior walls defining a lumen having a longitudinal axis;
a removable pull rod comprising a tip;
a piston located in the lumen of the syringe body, the piston together with the interior walls defining a working volume configured to hold fluid, the piston comprising a cavity configured to engage with the tip of the removable pull rod, wherein the piston is movable along the longitudinal axis to change the working volume; and
an insert narrowing the lumen to prevent retraction of the piston beyond a desired position;
a feedline configured to deliver droplets from the syringe for developing a solution for a test or a therapy; and
a syringe receptacle, the syringe receptacle configured to receive the syringe and fluidically couple the syringe to the feedline.

22. The system of claim 21, wherein the receptacle and the syringe are coupled by a snapping mechanism disposed at a distal end of the syringe.

23. The system of claim 21, wherein the receptacle and the syringe are coupled by screw-type mechanism, wherein the syringe includes threads formed at a proximal end on an outer surface of the syringe, and the receptacle includes corresponding threads at an interior portion of the receptacle.

24. The system of claim 21, wherein the receptacle includes a rubber septum to be penetrated by a needle attached at a proximal end of the syringe such that a tip of the needle enters the feedline to supply droplets into the feedline.

25. The system of claim 21, wherein the receptacle includes a plurality of hollow chambers, each chamber configured to receive one syringe.

26. The system of claim 25, wherein one or more hollow chambers of the receptacle are configured to receive one or more syringes of different sizes.

27. The system of claim 26, wherein the feedline has an inlet and an outlet, the receptacle being connected between the inlet and the outlet, wherein the inlet receives sterilized air, vacuum, or buffer from a pump and the outlet delivers fluids from the plurality of syringes in the receptacle.

28. The system of claim 27, further comprising:
a delivery mechanism configured to inject, at a specified time and a specified amount, fluid from one or more syringes in the receptacle.

29. The system of claim 21, wherein the tip is conical or spherical in shape having a narrow end configured to enter the cavity in the piston and a base end configured to couple the piston to the tip.

30. The system of claim 29, wherein the base end of the tip has a chamfered or fillet edge configured to facilitate retraction of the tip from the cavity of the piston.

31. The system of claim 29, wherein the piston is made of a compliant material.

32. The system of claim 31, wherein the cavity is cylindrical or spherical in shape and sized to provide a friction fit between the tip of the removable pull rod and the piston.

33. The system of claim 29, wherein the base end comprises a base portion configured to taper away from the narrow end.

34. The system of claim 33, wherein the cavity of the piston is configured to cover the base portion of the tip.

35. The system of claim 34, wherein the piston is made of a compliant material, wherein the cavity of the piston sized smaller than a size of the tip and is configured to conform to a shape of the tip of the removable pull rod, wherein when the piston receives the removable pull rod, the cavity of the piston expands around the tip thereby coupling the removable pull rod and the piston.

36. The system of claim 21, wherein the insert comprises a hollow cylinder defining an internal channel, wherein the internal channel is larger than an outside diameter of the removable pull rod and is configured to allow the removable pull rod to movably extend through the internal channel and move along the longitudinal axis, and to remove the removable pull rod from the syringe when decoupled from the piston.

37. A method of dispensing contents of a syringe, the method comprising:
filling a syringe by retracting a removable pull rod coupled to a piston located within a lumen of a syringe body;
separating the removable pull rod from the piston after filling the syringe;
inserting the syringe into a syringe receptacle to fluidically couple the syringe to a feedline;
coupling the removable pull rod to the piston; and
advancing the removable pull rod by a specified amount to move the piston within the lumen of the syringe body and thereby deliver a desired amount of fluid into the feedline.

38. The method of claim 37, further comprising coupling the syringe receptacle and the feedline.

39. The method of claim 37, wherein fluidically coupling the syringe to the feedline forms a sterile and functionally closed system.

40. A method of performing an assay, comprising:
delivering a sample from the syringe according to any preceding claim into a flow of fluid contained within a functionally-closed system; and
performing an assay.

41. The method of claim 40, wherein the sample comprises a gene editing reagent and the flow of fluid comprises a cell.
